(19) United States
(12) Patent Application Publication
(10) Pub. No.: US 2011/0319659 A1
(43) Pub. Date: Dec. 29, 2011

(54) METHOD FOR PRODUCING METHIONINE

(76) Inventors: Motonobu YOSHIKAWA, Saijo-shi (JP); Takehiro Nakasuji, Niihama-shi (JP)

(21) Appl. No.: 13/171,122

(22) Filed: Jun. 28, 2011

(30) Foreign Application Priority Data
Jun. 29, 2010 (JP) JP2010-148375

(51) Int. Cl. C07C 319/14

(52) U.S. Cl. 562/559

(57) ABSTRACT

A method for producing methionine includes a hydrolyzing step of hydrolyzing 5-(β-methylmercaptoethyl)hydantoin, and a crystallizing step of crystallizing with carbon dioxide introduced into a reaction solution after hydrolysis, to obtain methionine. In the crystallizing step, as carbon dioxide introduced into the hydrolysis reaction solution, carbon dioxide that is separated in a carbon dioxide separation section from a reformed gas formed by steam reforming reaction in a steam reformation section and carbon dioxide that is separated in an exhaust gas separation section from a combustion exhaust gas generated by pure oxygen combustion in a hydrocarbon heating furnace and a reformation reaction heating furnace are used.

### BACKGROUND OF THE INVENTION

1. Field of the Invention

The present invention relates to a method for producing methionine by utilizing carbon dioxide that is separated and recovered from a combustion exhaust gas obtained by pure oxygen combustion.

2. Description of the Related Art

There has been known a method of producing methionine by reacting 3-methylthiopropanal as a raw material with hydrocyanic acid in the presence of a base, then reacting the resultant with ammonium carbonate, and thereafter hydrolyzing the reaction product. In this method, carbon dioxide is introduced into the reaction liquid after hydrolysis, whereby crystallization occurs and methionine separated as a crystal can be obtained.

Carbon dioxide to be introduced into the reaction liquid after hydrolysis includes carbon dioxide that is generated in the process of producing hydrogen by the steam reforming reaction, and carbon dioxide that is obtained by rinsing and purifying an exhaust gas generated from a boiler or the like. Hydrogen is also used as a raw material for producing methionine, and therefore, the steam reforming reaction is generally utilized since a reformed gas containing hydrogen and carbon dioxide is generated thereby.

The amount ratio of hydrogen and carbon dioxide for producing methionine is hydrogen/carbon dioxide=1/1 in terms of molar ratio. On the other hand, the amount ratio of hydrogen and carbon dioxide generated by the steam reforming reaction is approximately hydrogen/carbon dioxide=3/1 in terms of molar ratio. Accordingly, assuming that hydrogen and carbon dioxide contained in the reformed gas formed by the steam reforming reaction are used as raw materials for producing methionine, hydrogen is left over, and facilities for processing the excessive hydrogen are necessarily provided.

Japanese Unexamined Patent Publication JP-A 2003-81605 discloses a method for producing hydrogen, in which hydrogen is separated and purified from a reformed gas formed by subjecting liquefied natural gas to steam reforming reaction, and an offgas containing combustible substances separated in the purification process of hydrogen is used for combustion for heating in the steam reforming reaction. In the method for producing hydrogen disclosed in JP-A 2003-81605, pure oxygen or high-concentration oxygen, which is cryogenically separated by utilizing liquefaction heat of liquefied natural gas, is introduced as an oxidizing agent for combustion of the offgas on heating for combustion in the steam reforming reaction, and carbon dioxide is separated and recovered at a high concentration from the combustion exhaust gas generated on the combustion. In the method for producing hydrogen, carbon dioxide in the reformed gas formed by the steam reforming reaction and high-concentration carbon dioxide separated and recovered from the combustion exhaust gas are obtained.

However, JP-A 2003-81605 does not consider the use of hydrogen and carbon dioxide formed and recovered in producing hydrogen, as raw materials for producing methionine. Therefore, hydrogen is left over for the production of methionine where hydrogen and carbon dioxide are used at a molar ratio of hydrogen/carbon dioxide=1/1, and facilities for processing the excessive hydrogen is still necessarily provided.

### SUMMARY OF THE INVENTION

Hence, an object of the invention is to provide a method for producing methionine by utilizing hydrogen and carbon dioxide that is formed and recovered in producing hydrogen, the method being capable of decreasing the amount of excessive hydrogen.

The invention provides a method for producing methionine, comprising:
a hydantoin step of obtaining 5-(β-methylmercaptoethyl)hydantoin by using hydrogen sulfide obtained through reaction of hydrogen and sulfur;
a hydrolyzing step of hydrolyzing 5-(β-methylmercaptoethyl)hydantoin;
a crystallizing step of crystallizing with carbon dioxide introduced into a reaction solution after hydrolysis, to obtain methionine; and
a raw material feeding step of feeding hydrogen and carbon dioxide that are formed and recovered from a hydrogen producing apparatus, in which a reformed gas is formed by subjecting a hydrocarbon heated with a heating furnace and steam to steam reforming reaction under heating by combustion, as hydrogen for use in the hydantoin step and carbon dioxide for use in the crystallizing step,
in the raw material feeding step,
hydrogen that is separated and recovered from the reformed gas formed in the hydrogen producing apparatus being fed for use in the hydantoin step,
carbon dioxide that is separated and recovered from the reformed gas formed in the hydrogen producing apparatus being fed for use in the crystallizing step as main-material carbon dioxide, and
carbon dioxide that is separated and recovered from a combustion exhaust gas generated in combustion in the heating furnace for heating the hydrocarbon, and carbon dioxide that is separated and recovered from a combustion exhaust gas generated in combustion for heating in the steam reforming reaction with oxygen obtained by cryogenic separation of air introduced as an oxidizing agent, being fed for use in the crystallizing step as auxiliary-material carbon dioxide.

According to the invention, the method for producing methionine comprises: a hydantoin step of obtaining 5-(β-methylmercaptoethyl)hydantoin by using hydrogen sulfide obtained through reaction of hydrogen and sulfur; a hydrolyzing step of hydrolyzing 5-(β-methylmercaptoethyl)hydantoin; a crystallizing step of crystallizing with carbon dioxide introduced into a reaction solution after hydrolysis, to obtain methionine; and a raw material feeding step.

In the raw material feeding step, hydrogen that is separated and recovered from the reformed gas formed in the hydrogen producing apparatus is fed for use in the hydantoin step. In the raw material feeding step, furthermore, carbon dioxide that is separated and recovered from the reformed gas formed in the hydrogen producing apparatus is fed for use in the crystallizing step as main-material carbon dioxide, and carbon dioxide that is separated from a combustion exhaust gas generated in combustion in the heating furnace for heating the hydrocarbon, and carbon dioxide that is separated from a combustion exhaust gas generated in combustion for heating in the steam reforming reaction with pure oxygen or high-concentration oxygen obtained by cryogenic separation of air introduced as an oxidizing agent (pure oxygen combustion), are fed for use in the crystallizing step as auxiliary-material carbon dioxide.

Further, in the method for producing methionine of the invention, it is preferable that the combustion in the heating furnace for heating the hydrocarbon in the hydrogen producing apparatus is combustion with oxygen obtained by cryogenic separation of air introduced as an oxidizing agent.

Further, in the method for producing methionine of the invention, the combustion in the heating furnace for heating the hydrocarbon in the hydrogen producing apparatus is preferably combustion with pure oxygen or high-concentration oxygen obtained by cryogenic separation of air introduced as an oxidizing agent (pure oxygen combustion).

Further, in the method for producing methionine of the invention, it is preferable that steam for use in the steam reforming reaction is generated by utilizing heat energy of the reformed gas of the steam reforming reaction in the hydrogen producing apparatus.

According to the invention, in the method for producing methionine, methionine is produced by utilizing hydrogen and carbon dioxide obtained from the hydrogen producing apparatus at a molar ratio of hydrogen/carbon dioxide=1/1, which are constituted by hydrogen and carbon dioxide that are separated and recovered from the reformed gas formed by the steam reforming reaction (main-material carbon dioxide) and high-concentration carbon dioxide that is separated and recovered from the combustion exhaust gas by the pure hydrogen combustion (auxiliary-material carbon dioxide), and therefore, the amount of excessive hydrogen can be decreased.

According to the invention, in the method for producing methionine, steam for use in the steam reforming reaction is generated by utilizing heat energy of the reformed gas of the steam reforming reaction. Accordingly, in a case of obtaining hydrogen and carbon dioxide at a molar ratio of hydrogen/carbon dioxide=1/1 in the hydrogen producing apparatus, surplus heat energy over the quantity that is required for the steam reforming reaction can be recovered as steam.

### BRIEF DESCRIPTION OF THE DRAWINGS

Other and further objects, features, and advantages of the invention will be more explicit from the following detailed description taken with reference to the drawings wherein:

FIG. 1 is a diagram showing a configuration of a hydrogen producing apparatus which generates carbon dioxide and hydrogen, used in a method for producing methionine according to an embodiment of the invention.

### DETAILED DESCRIPTION

Now referring to the drawings, preferred embodiments of the invention are described below.

A method for producing methionine of the invention is a method for producing ethionine by utilizing hydrogen and carbon dioxide that are formed and recovered in producing hydrogen, and the method comprises a hydantoin step, a hydrolyzing step and a crystallizing step. The hydantoin step includes a hydrogen sulfide forming step, a methylmercaptan forming step, an acrolein forming step, a methylthiopropanal forming step, a cyanohydrin forming step and a hydantoin forming step.

In the hydrogen sulfide forming step, hydrogen sulfide (H₂S) is obtained by reacting hydrogen (H₂) and sulfur (S) as shown in the following equation (1). Hydrogen is used in the hydrogen sulfide forming step.

H₂+S→H₂S (1)

In the methylmercaptan forming step, methylmercaptan (CH₃SH) is obtained by reaction shown in the following equations (2), (3) and (4). In the equations (2), (3) and (4), CH₃OH shows methanol, and CH₃SCH₃ shows dimethylsulfide.

H₂S+CH₃OH→CH₃SH+H₂O (2)

CH₃OH+CH₃SH→CH₃SCH₃+H₂O (3)

CH₃SCH₃+H₂S [symbol] 2CH₃SH (4)

In the acrolein forming step, acrolein (CH₂=CHCHO) is obtained by reacting propylene (CH₂=CHCH₃) and oxygen (O₂) as shown in the following equation (5).

CH₂=CHCH₃+O₂→CH₂=CHCHO+H₂O (5)

In the methylthiopropanal forming step, 3-methylthiopropanal is obtained by reacting acrolein and methylmercaptan as shown in the following equation (6).

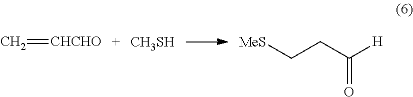

In the cyanohydrin forming step, 2-hydroxy-4-methylthiobutanenitrile is obtained by reacting 3-methylthiopropanal and hydrocyanic acid (HCN) as shown in the following equation (7).

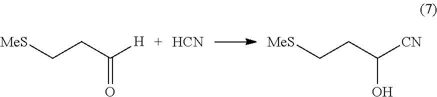

In the hydantoin forming step, 5-(β-methylmercaptoethyl)hydantoin is obtained by reacting 2-hydroxy-4-methylthiobutanenitrile and ammonium carbonate as shown in the following equation (8). In the hydantoin forming step, ammonium carbonate may be used as it is or may be used in the form of an aqueous solution of ammonium carbonate. Furthermore, ammonium carbonate may be prepared from carbon dioxide gas and ammonia in the reaction system or in the solvent, or in alternative, ammonium carbonate prepared from ammonium bicarbonate and potassium hydroxide may be used.

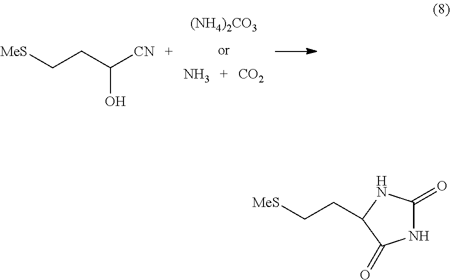

In the hydrolyzing step, 5-(β-methylmercaptoethyl)hydantoin is hydrolyzed in the presence of a basic potassium compound, thereby obtaining methionine, as shown in the following equation (9). Examples of the basic potassium compound include potassium hydroxide, potassium carbonate and potassium hydrogen carbonate, two or more kinds of which may be used depending on necessity. The hydrolyzing reaction may be performed in water, and methionine is present in the form of a potassium salt in the resulting hydrolysis reaction solution.

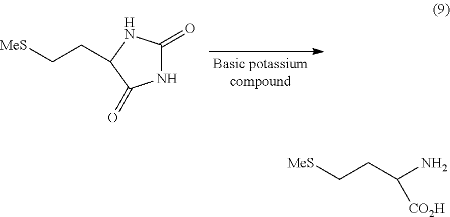

In the crystallizing step, crystallization is performed with carbon dioxide introduced into the reaction solution, for collecting methionine present in the form of a potassium salt in the hydrolysis reaction solution, and the resulting slurry is separated into a deposited product and a mother liquid by filtration, decantation or the like, thereby obtaining methionine in the form of crystals. The reaction solution absorbs carbon dioxide through introduction of carbon dioxide, and free methionine is deposited from the potassium salt of methionine. Carbon dioxide is used in the crystallizing step.

Methionine thus separated may be subjected to rinsing, pH adjustment and the like as necessary, and then dried to provide a product.

In the method for producing methionine according to the embodiment, methionine is produced by utilizing hydrogen and carbon dioxide formed and recovered in producing hydrogen.

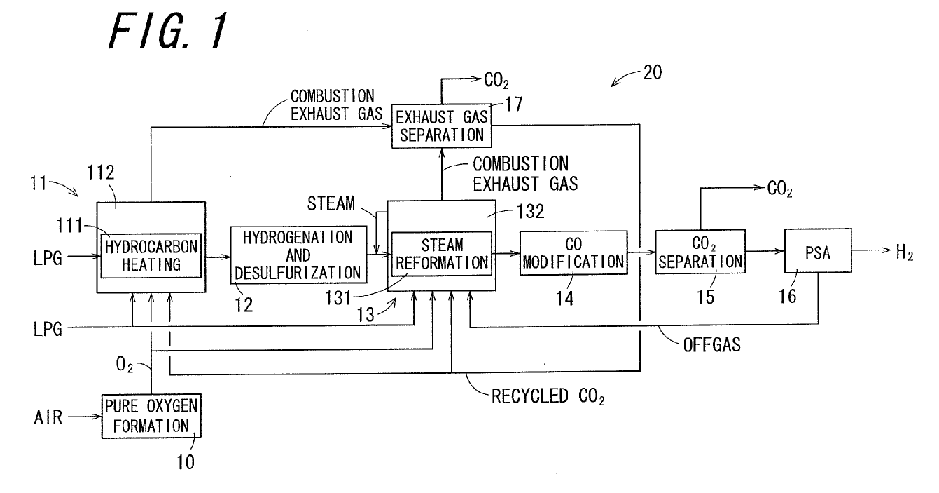

FIG. 1 is a diagram showing a configuration of a hydrogen producing apparatus 20 which generates carbon dioxide and hydrogen, used in the method for producing methionine according to the embodiment of the invention. The hydrogen producing apparatus 20 produces hydrogen by performing steam reforming reaction with a hydrocarbon and steam as raw materials. In the method for producing methionine according to the embodiment, the raw material feeding step is implemented with the hydrogen producing apparatus 20.

Examples of the hydrocarbon include natural gas containing methane as a major component, liquefied petroleum gas (LPG), liquefied natural gas (LNG) and naphtha, and in the embodiment, LPG is used. The hydrocarbon preferably has a low sulfur concentration in consideration of reduction of contamination in the crystallizing step.

The hydrogen producing apparatus 20 comprises a cryogenic air separation section 10, a hydrocarbon heating section 11 comprising a hydrocarbon heating device 111 and a hydrocarbon heating furnace 112, a hydrogenation and desulfurization section 12, a steam reformation section 13 comprising a reformation reactor 131 and a reformation reaction heating furnace 132, a carbon monoxide modification section 14, a carbon dioxide separation section 15, a purification section 16, and an exhaust gas separation section 17.

The cryogenic air separation section 10 subjects air as a raw material to cryogenic separation to form pure oxygen or high-concentration oxygen. Oxygen that is obtained by the cryogenic air separation (which may be hereinafter referred to as “cryogenic air separation oxygen”) is fed to the hydrocarbon heating furnace 112 of the hydrocarbon heating section 11 and the reformation reaction heating furnace 132 of the steam reformation section 13, and used as an oxidizing agent for combustion for heating.

The hydrocarbon heating section 11 comprises the hydrocarbon heating device 111 and the hydrocarbon heating furnace 112, and heats LPG as a raw material of the steam reforming reaction (which may be hereinafter referred to as “reaction material LPG”). The reaction material LPG is fed to the hydrocarbon heating device 111, and the reaction material LPG thus fed is heated, for example, to 620° C. with heat energy of combustion in the hydrocarbon heating furnace 112. The reaction material LPG thus heated is fed to the hydrogenation and desulfurization section 12.

LPG as a combustion fuel (which may be hereinafter referred to as “combustion fuel LPG”), the cryogenic air separation oxygen as an oxidizing agent from the cryogenic air separation section 10, and carbon dioxide as a diluent from the exhaust gas separation section 17 (which may be hereinafter referred to as “recycled carbon dioxide”) are fed to the hydrocarbon heating furnace 112. In the hydrocarbon heating furnace 112, for example, the combustion fuel LPG is fed at 67.4 kg/H (1.16 kmol/H), the cryogenic air separation oxygen is fed at 180 Nm³/H, and the recycled carbon dioxide (temperature: 225° C.) is fed at 635 Nm³/H, thereby performing combustion with the cryogenic air separation oxygen introduced as an oxidizing agent (pure oxygen combustion). A combustion exhaust gas is generated by the pure oxygen combustion, and carbon dioxide is generated at 4.6 kmol/H (104 Nm³/H) as a component of the combustion exhaust gas. The combustion exhaust gas generated from the hydrocarbon heating furnace 112 is fed to the exhaust gas separation section 17.

The hydrogenation and desulfurization section 12 subjects the reaction material LPG, which has been heated in the hydrocarbon heating section 11, to hydrogenation and desulfurization. The reaction material LPG having been subjected to hydrogenation and desulfurization is fed to the reformation reactor 131 of the steam reformation section 13.

The steam reformation section 13 comprises the reformation reactor 131 and the reformation reaction heating furnace 132, and performs steam reforming reaction. The reformation reactor 131 performs steam reforming reaction with the reaction material LPG fed from the hydrogenation and desulfurization section 12 and steam as raw materials. The steam reforming reaction performed in the reformation reactor 131 is performed in the presence of a reformation catalyst such as a Ni (nickel) series or Ru (ruthenium) series catalyst, in the reformation reaction heating furnace 132 at a high temperature of from 500° C. to 1000° C., preferably from 800° C. to 1000° C. (which is 850° C. in the embodiment), under a high pressure of approximately from 0.5 MPa to 3.5 MPa. When the temperature on the steam reforming reaction exceeds 1000° C., an aromatic hydrocarbon is unfavorably formed in the vicinity of the wall of the reformation reactor 131.

In the reformation reactor 131, the steam reforming reaction performed generates a reformed gas containing hydrogen, carbon monoxide and carbon dioxide as generated gases, and the hydrocarbon and steam as unreacted gases. The reformed gas in the reformation reactor 131 is fed to the carbon monoxide modification section 14.

In the reformation reactor 131, for example, the reaction material LPG is fed at 58.5 kmol/H, thereby performing the steam reforming reaction. The steam reforming reaction generates a reformed gas, and as components of the reformed gas, hydrogen is generated at 760.5 kmol/H, and carbon dioxide is generated at 234.0 kmol/H.

Further, in the method for producing methionine according to the embodiment, steam for use in the steam reforming reaction is generated by utilizing heat energy of the reformed gas of the steam reforming reaction in the reformation reactor 131. Accordingly, in a case of obtaining hydrogen and carbon dioxide at a molar ratio of hydrogen/carbon dioxide=1/1 in the hydrogen producing apparatus 20, surplus heat energy over the quantity that is required for the steam reforming reaction can be recovered as steam.

The combustion fuel LPG, the cryogenic air separation oxygen as an oxidizing agent from the cryogenic air separation section 10, the recycled carbon dioxide as a diluent from the exhaust gas separation section 17, and an offgas (which contains hydrogen, methane, carbon monoxide, carbon dioxide and the like) from the purification section 16 are fed to the reformation reaction heating furnace 132. In the reformation reaction heating furnace 132, for example, the combustion fuel LPG is fed at 4123 kg/H (70.93 kmol/H), the cryogenic air separation oxygen is fed at 12252 Nm³/H, the recycled carbon dioxide (temperature: 225° C.) is fed at 113560 Nm³/H, and the offgas is fed at 3700 Nm³/H, thereby performing combustion with cryogenic air separation oxygen introduced as an oxidizing agent (pure oxygen combustion). A combustion exhaust gas is generated by the pure oxygen combustion, and carbon dioxide is generated at 356.4 kmol/H (7983 Nm³/H) as a component of the combustion exhaust gas. The combustion exhaust gas generated from the reformation reaction heating furnace 132 is fed to the exhaust gas separation section 17.

The carbon monoxide modification section 14 converts carbon monoxide contained in the reformed gas fed from the reformation reactor 131 to carbon dioxide. The carbon monoxide modification section 14 comprises a high-temperature modification section and a low-temperature modification section. In the high-temperature modification section, conversion reaction is performed in the presence of an iron-chromium oxide catalyst to decrease the carbon monoxide concentration in the reformed gas, and in the low-temperature modification section, conversion reaction is performed in the presence of a copper-zinc oxide catalyst to decrease further the carbon monoxide concentration in the reformed gas. A heat exchanger which performs exchange of heat is disposed between the high-temperature modification section and the low-temperature modification section. The reformed gas discharged from the carbon monoxide modification section 14 is fed to the carbon dioxide separation section 15.

The carbon dioxide separation section 15 separates and recovers carbon dioxide from the reformed gas fed from the carbon monoxide modification section 14. Carbon dioxide thus separated and recovered by the carbon dioxide separation section 15 is fed into the hydrolysis reaction solution, as main-material carbon dioxide for use in the crystallizing step.

The reformed gas is generated by the steam reforming reaction in the reformation reactor 131, and carbon dioxide is generated as a component of the reformed gas at 234.0 kmol/H, as described above. The carbon dioxide separation section 15 separates and recovers carbon dioxide from the reformed gas. Specifically, the carbon dioxide separation section 15 separates and recovers carbon dioxide at 171.3 kmol/H (3837 Nm³/H) from the reformed gas. The reformed gas discharged from the carbon dioxide separation section 15 is fed to the purification section 16.

The purification section 16 separates and recovers hydrogen from the reformed gas fed from the carbon dioxide separation section 15. Hydrogen thus separated and recovered by the purification section 16 is fed as hydrogen for use in the hydrogen sulfide forming step of the hydantoin step.

The purification section 16 may have a structure where hydrogen is separated with an adsorbent by pressure swing adsorption or temperature swing adsorption, or a structure using a hydrogen separation membrane that selectively transmits hydrogen only. The purification section 16 in the embodiment has a structure where hydrogen is separated by pressure swing adsorption (PSA). The reformed gas is generated by the steam reforming reaction in the reformation reactor 131, and hydrogen is generated as a component of the reformed gas at 760.5 kmol/H, as described above. The purification section 16 separates and recovers hydrogen from the reformed gas. Specifically, the purification section 16 separates and recovers hydrogen at 532.3 kmol/H (11924 Nm³/H) from the reformed gas.

The exhaust gas separation section 17 separates and recovers carbon dioxide from the combustion exhaust gas generated by the pure oxygen combustion, which is fed from the hydrocarbon heating furnace 112 and the reformation reaction heating furnace 132. Carbon dioxide thus separated and recovered by the exhaust gas separation section 17 is fed into the hydrolysis reaction solution, as auxiliary-material carbon dioxide for use in the crystallizing step.

The combustion exhaust gas is generated by the pure oxygen combustion in the hydrocarbon heating furnace 112, and carbon dioxide is generated at 4.6 kmol/H (104 Nm³/H) as a component of the combustion exhaust gas, as described above. The combustion exhaust gas is generated by the pure oxygen combustion in the reformation reaction heating furnace 132, and carbon dioxide is generated at 356.4 kmol/H (7983 Nm³/H) as a component of the combustion exhaust gas, as described above. The exhaust gas separation section 17 separates and recovers carbon dioxide from the combustion exhaust gas generated by the pure oxygen combustion in the hydrocarbon heating furnace 112 and the reformation reaction heating furnace 132. Specifically, the exhaust gas separation section 17 separates and recovers carbon dioxide at 361.0 kmol/H (8087 Nm³/H) from the combustion exhaust gas in the hydrocarbon heating furnace 112 and the reformation reaction heating furnace 132.

In the hydrogen producing apparatus 20 having the aforementioned structure, the feeding amount of the reaction raw material LPG for use in the steam reforming reaction in the reformation reactor 131, and the feeding amounts of the combustion fuel LPG, the cryogenic air separation oxygen, the recycled carbon dioxide and the offgas for use in the pure oxygen combustion in the hydrocarbon heating furnace 112 and the reformation reaction heating furnace 132 are so controlled that the amount of hydrogen recovered in the purification section 16 (532.3 kmol/H) and the total amount of carbon dioxide recovered in the carbon dioxide separation section 15 and the exhaust gas separation section 17 (171.3+361.0=532.3 kmol/H) have a molar ratio of hydrogen/carbon dioxide=1/1.

In the crystallizing step of the method for producing methionine according to the embodiment, as carbon dioxide introduced into the hydrolysis reaction solution, carbon dioxide that is separated in the carbon dioxide separation section 15 from the reformed gas formed by the steam reforming reaction in the steam reformation section 13 (main-material carbon dioxide), and carbon dioxide that is separated in the exhaust gas separation section 17 from the combustion exhaust gas generated by the pure oxygen combustion with cryogenic air separation oxygen obtained in the cryogenic air separation section 10 introduced as an oxidizing agent in the hydrocarbon heating furnace 112 and the reformation reaction heating furnace 132 (auxiliary-material carbon dioxide) are used.

In the method for producing methionine according to the embodiment, methionine is produced by utilizing hydrogen and carbon dioxide obtained from the hydrogen producing apparatus 20 at a molar ratio of hydrogen/carbon dioxide=1/1, which are constituted by hydrogen and carbon dioxide that are formed by the steam reforming reaction (main-material carbon dioxide) and high-concentration carbon dioxide that is separated and recovered from the combustion exhaust gas by the pure hydrogen combustion (auxiliary-material carbon dioxide), and therefore, the amount of excessive hydrogen can be decreased.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

What is claimed is:

1. A method for producing methionine, comprising:
a hydantoin step of obtaining 5-(β-methylmercaptoethyl)hydantoin by using hydrogen sulfide obtained through reaction of hydrogen and sulfur;
a hydrolyzing step of hydrolyzing 5-(β-methylmercaptoethyl)hydantoin;
a crystallizing step of crystallizing with carbon dioxide introduced into a reaction solution after hydrolysis, to obtain methionine; and
a raw material feeding step of feeding hydrogen and carbon dioxide that are formed and recovered from a hydrogen producing apparatus, in which a reformed gas is formed by subjecting a hydrocarbon heated with a heating furnace and steam to steam reforming reaction under heating by combustion, as hydrogen for use in the hydantoin step and carbon dioxide for use in the crystallizing step,
in the raw material feeding step,
hydrogen that is separated and recovered from the reformed gas formed in the hydrogen producing apparatus being fed for use in the hydantoin step,
carbon dioxide that is separated and recovered from the reformed gas formed in the hydrogen producing apparatus being fed for use in the crystallizing step as main-material carbon dioxide, and
carbon dioxide that is separated and recovered from a combustion exhaust gas generated in combustion in the heating furnace for heating the hydrocarbon, and carbon dioxide that is separated and recovered from a combustion exhaust gas generated in combustion for heating in the steam reforming reaction with oxygen obtained by cryogenic separation of air introduced as an oxidizing agent, being fed for use in the crystallizing step as auxiliary-material carbon dioxide.

2. The method for producing methionine of claim 1, wherein the combustion in the heating furnace for heating the hydrocarbon in the hydrogen producing apparatus is combustion with oxygen obtained by cryogenic separation of air introduced as an oxidizing agent.

3. The method for producing methionine of claim 1, wherein steam for use in the steam reforming reaction is generated by utilizing heat energy of the reformed gas of the steam reforming reaction in the hydrogen producing apparatus.

4. The method for producing methionine of claim 2, wherein steam for use in the steam reforming reaction is generated by utilizing heat energy of the reformed gas of the steam reforming reaction in the hydrogen producing apparatus.
